(19) United States
(12) Patent Application Publication
(10) Pub. No.: US 2024/0081804 A1
(43) Pub. Date: Mar. 14, 2024

(54) Surgical Implant for Reinforcing Soft Tissue Repair Such as Rotator Cuff Tendon

(71) Applicant: Michael Francis, Norfolk, VA (US)

(72) Inventor: Michael Francis, Norfolk, VA (US)

(21) Appl. No.: 18/465,255

(22) Filed: Sep. 12, 2023

Related U.S. Application Data
(60) Provisional application No. 63/375,690, filed on Sep. 14, 2022.

(51) Int. Cl. A61B 17/04; A61F 2/08

(52) U.S. Cl. CPC A61B 17/0401; A61F 2/0811; A61B 2017/0409; A61B 2017/0438; A61B 2017/0464; A61F 2002/0817; A61F 2002/0858

(57) ABSTRACT

A soft tissue augmentation implant for surgically reinforcing soft tissue, such as a rotator cuff tendon in the shoulder. The implant comprises one or more slots for passing a suture through the implant. In a two-slot design, the suture may pass through the slots in a buckle configuration. The suture secures the implant to the target soft tissue. The bottom side of the implant has a rough surface or has one or protrusions. This feature facilitates mechanical integration of the implant to the target soft tissue. Also disclosed are soft tissue augmentation assemblies, surgical kits, and methods for reinforcing soft tissue (such as rotator cuff repair).

### TECHNICAL FIELD

This invention relates to surgical implants for soft tissue repair, such as rotator cuff tendon repair.

### BACKGROUND

Rotator cuff repair is a surgical procedure that is commonly performed on shoulders. Many conventional techniques use sutures only. FIGS. 1A and 1B show an example of a conventional suture-only technique for repairing a torn supraspinatus tendon of the rotator cuff. In FIG. 1A, the relevant anatomy includes the head 12 of the humerus bone, an injured supraspinatus tendon 10, and joint capsule 13. Here, supraspinatus tendon 10 is reinforced by a conventional double-row repair technique using tape sutures 16. These are anchored to suture anchors 14. FIG. 1B shows how this suture-only repair can fail at the suture-tendon interface. Here, there is a tear 18 where suture 16 has cut through supraspinatus tendon 10. This is one of the leading causes of rotator cuff repair failure.

Other surgical products for enhancing rotator cuff repair have been developed. For example, tissue engineered patches are designed to promote tissue ingrowth into the patch and remodeling at the site of the tendon injury. However, such patches are highly porous and mechanically weak when hydrated. They are not designed for load sharing or augmenting the strength of the tendon repair. Thus, these patch products do not directly address tendon-suture failure in rotator cuff repair.

### SUMMARY

In one aspect, this invention is a soft tissue augmentation implant for reinforcing soft tissue. For example, the augmentation implant could be used in repairing a rotator cuff tendon. Among the possible advantages of this invention are better distribution of the suturing load, reducing stress and strain on the repair site, reduced costs, improved success rates, and reduced post-repair failures (such as repeat tearing).

Soft Tissue Implant. The soft tissue augmentation implant comprises a main body that has a generally flat block-like shape. From an overhead top view, the augmentation implant could have any suitable shape, such as trapezoidal, square, rectangular, ovoid, round, etc. The augmentation implant has one, two, three, four, or more slots for receiving a suture therethrough. The slot(s) are through-holes in the implant body and could have any suitable shape such as rectangular, square, round, ovoid, triangular, etc. In another example, the flat body is “L-shaped” with one, two, three, or more slots in each leg for the suture that is directed along multiple directions; such as in double-row rotator cuff repair, or for mattress or whip stitches (such as in Achilles tendon, fascia, muscle, and other soft tissue repairs).

The slot(s) may be positioned to receive a single suture, or designed to receive two or more sutures. The augmentation implant could be made of any suitable material, including polymer materials in any suitable form (e.g. as bulk solid or as fibers). Examples of polymer materials that could be used include polydioxanone, polyethylene (such as high-density polyethylene, HDPE), polycaprolactone, nylon, polypropylene, polytetrafluoroethylene (PTFE), polymeric D-lactic acid (PDLA), polymeric L-lactic acid (PLLA), copolymeric lactide/glycolide (PLGA), collagen, cellulose, nanocellulose, chitosan, silk, microfibrillated cellulose, etc. In some cases, the implant does not comprise any polydioxanone (PDO) polymer. The implant could be permanent or resorbable (bioabsorbable). The implant may comprise a biodegradable polymer. The implant could have a bioactive or biologic coating (such as collagen) to promote healing or integration.

The augmentation implant could have void features such as pockets, underpass tunnels, or notches. For example, there could be an underpass tunnel on the bottom side. This underpass tunnel could extend from an entrance/exit on an edge side (e.g. lateral or medial). This underpass tunnel could extend to the medial slot only or to both slots (in relevant design embodiments). In another example, there could be an overpass pocket on the top side. In another example, there could be a notch on the lateral or medial side.

The augmentation implant could be non-porous. This distinguishes from other types of soft tissue implants that are porous to facilitate tissue integration. The implant is substantially rigid and minimally flexible. This distinguishes from flexible patch implants that made of other biomaterials, such as electrospun or lyophilized collagen. The slot(s) could have teeth to help grip the suture. The implant could be medial-lateral asymmetric.

Dimensions. The main body of the augmentation implant has a width of 2-20 mm; and in some cases, 3-8 mm. Having a narrower width could be useful for facilitating arthroscopic deployment of the implant to the surgical site through the bore of a surgical cannula. For shapes where length is a relevant dimension, the main body could have a length of 2-20 mm; and in some cases, 5-12 mm. The main body could have a thickness of 0.1-5.0 mm; and in some cases, 0.6-3.0 mm. The top surface of the main body could have a surface area of 10-200 mm²; and in some cases, 25-80 mm².

The main body of the augmentation implant could have a generally tapered shape (not counting any protrusions on the bottom side) along the longitudinal axis. The thickness of the main body may decrease going from lateral to medial. The thickness of the main body at a lateral point of measurement may be greater than the thickness at a medial point of measurement.

Bottom Surface Texture, Roughness or Protrusions. The top surface may be smooth and featureless. This may be useful in inhibiting tissue adhesion. In contrast, the bottom surface may be non-smooth (textured) or rough. In relative terms, the bottom surface could be defined as being less smooth than the top surface; or vice versa, the top surface is smoother than the bottom surface. Alternately, the bottom surface could be defined as being rougher than the top surface. For example, the bottom surface could have a general coarseness.

There are a variety of designs that could give the bottom surface a non-smooth (textured) or rough characteristic. The purpose of this design feature is to facilitate the mechanical hold of the augmentation implant with the underlying tissue and the subsequent integration of the augmentation implant. In some embodiments, the bottom surface has protruding features such as teeth, ledges, grapples, hooks, barbs, ridges, bumps, knobs, knurls, etc. These lengths may be in the micrometers to millimeters range. The protruding features may pierce into the underlying body tissue. The downward length of the protruding feature could be at least 1.5 mm or in the range of 0.5-5 mm; and could be longer than the thickness of the main body. Note that if the augmentation implant has protruding features, those are not counted as part of the measurement for the thickness of the main body.

The bottom surface could have recessed features, such as holes, cavities, grooves, clefts, dimples, etc. For example, the bottom surface could be fenestrated with multiple small holes having a size of 10-500 μm (such an array of such holes) or have scored lines. These holes would promote tissue growth therein. The bottom surface could have a combination of protruding and recessed features. For example, some feature patterns have both protruding and recessed elements. For example, knurls are alternating ridges and grooves in a cross-grained pattern. Note that if the augmentation implant has recessed features, those are not counted as part of the measurement for the thickness of the flat body. The top surface could also be non-smooth (textured) or rough. This may promote holding of the suture or facilitate surgical delivery.

Soft Tissue Augmentation Assembly. In another aspect, this invention is a soft tissue augmentation assembly. The assembly comprises the soft tissue augmentation implant described herein and a suture. The suture could be a round or flat tape-type suture having a width of 0.5-3.0 mm. The width of the suture may be narrower than the width of the slot(s) in the augmentation implant. The suture is assembled together with the implant by passing through the slot(s) of the augmentation implant. The suture may travel through the slot(s) in any suitable manner. See below section on ‘Treatment Method’ for more details about this. As assembled, the augmentation implant could be placed onto the target soft tissue (e.g. rotator cuff tendon, abdominal fascia in hernia repair, or torn Achilles tendon) and be secured thereto by anchoring of the suture.

Soft Tissue Augmentation Kit. In another aspect, this invention is a surgical kit product for surgically reinforcing soft tissue. The surgical kit comprises the soft tissue augmentation implant described herein and a suture. In this surgical kit, the components are provided together in the same package. The surgical kit may further include a device to load the suture and deploy the augmentation implant along the suture for arthroscopic placement in the surgical field, such as at the tendon-suture interface in rotator cuff repair surgery. The suture could be a separate component or could be provided already tethered to the implant. The surgical kit may further include a suture anchor (e.g. for anchoring into bone or soft tissue). The suture anchor could be a separate component or could be provided already pre-fastened to a suture.

Treatment Method. In another aspect, this invention is a method of surgically reinforcing soft tissue, such as muscles or fibrous connective tissue (e.g. tendons, fascia, ligament, etc.). One particular example of targeted soft tissue is a rotator cuff tendon in the shoulder. Another example is a breached fascia resulting in a hernia. Another example is chest sternum closure (e.g. in cardiac or thoracic surgery). The method uses the soft tissue augmentation implant of this invention. In the context of the shoulder joint, medial means towards the neck and lateral means outward towards the right or left side. This medial/lateral orientation could also apply to other surgical scenarios. The steps of this treatment method described below could be performed in any suitable order, and in some situations, certain steps can be omitted.

Anchor the suture at a first anchor site. Pass the suture through the one or more slots of the implant. Anchor the suture to a second anchor site. The second anchor site may be located lateral to the first anchor site (e.g. the first anchor site is medial and the second anchor site is lateral). Secure the implant on the target soft tissue. The steps of anchoring the suture and passing the suture through the slot(s) could be performed in any suitable order. For example (for a two-slot implant), pass the suture through one slot (e.g. medial slot) outside the surgical site, then place the implant into the surgical site, then anchor the suture at the first anchor site, then pass the suture through the other slot (e.g. lateral slot), and then anchor the suture at the second anchor site. Other possible variations in the order of steps are explained below.

The anchor sites could be located in various suitable positions relative to the implant, such as lateral to, medial to, or under the implant. The second anchor site could be located on an opposite side of the implant relative to the first anchor site. The first anchor site could be located medial to the implant and the second anchor site could be located lateral to the implant. In certain cases, such as rotator cuff repair, the medially-located first anchor site is closer to the augmentation implant than the laterally-located second anchor site.

The suture could pass through the slot(s) in any suitable path. For example, for implants having two slots, the suture could pass through the implant in a buckle configuration. For example (for a two-slot implant), the suture could travel over one edge of the implant, down through the first slot, back up the second slot, and then over the opposite edge of the implant (i.e. over-under-over path). Alternately, the suture could travel under one edge of the implant, up through the first slot, back down the second slot, and then under the opposite edge of the implant (i.e. under-over-under path). This buckle configuration gives the implant a tight grip on the suture.

Inserting the suture into the slot(s) may be performed internally within the surgical site, external to the surgical site (e.g. on a tray at the surgical bedside before inserting the implant into the surgical site by arthroscope and then shuttled down to the internal surgical site), or a combination thereof (e.g. the suture is passed through one slot externally at the surgical bedside and then passed through the other slot within the internal surgical site). The procedure could be performed by arthroscopic surgery. For example, insert a surgical cannula into the surgical site and deliver the implant through the bore of the surgical cannula.

The implant could be secured to the target soft tissue in any suitable orientation or position. The following examples are for a two-slot implant. For example, in the case of tendons, orient the implant such that its longitudinal axis or slots are aligned substantially orthogonal to the direction of the tendon fibers. For example, for treating rotator cuff tendons, orient the implant such that the slots are aligned substantially parallel to the anatomical A-P axis (anterior-posterior). In another example, secure the implant at a position such that the medial slot is positioned directly over the medial anchor site, and the suture travels upward from the medial anchor site and passes through the medial slot.

The anchor sites may be suture anchors that are embedded at the surgical site (e.g. into bone or soft tissue). The suture anchors could be embedded using any suitable surgical technique such as drilling a socket or directly punching the suture anchor into the bone with a punching instrument. A suture anchor could be provided with the suture already attached thereto (i.e. a combined assembly). In such cases, the step of fastening the suture to the anchor may be omitted. The implant could be provided with the suture already inserted through one or more of the slots (e.g. as pre-loaded assembly with the suture). In such cases, one or more steps of inserting the suture through the slot(s) may be omitted. The implant could also be provided pre-attached either impermanently or permanently to the suture, with the suture clamped together, molded together, passed through a slot, or otherwise formed together to produce an implant tethered to the suture or similar tissue-joining surgical accessory.

Miscellaneous Aspects. The following are some additional embodiments of this invention. Two or more of the implants may be placed side-by-side in a row for more footprint coverage on the target soft tissue. The sutures may run in parallel or non-parallel to either the same or different tissue fixation points. There may be no knots made in the suture at the suture-tissue interface. The treatment site may be a rotator cuff tendon at the shoulder joint and the bone is the humerus. The segment length of the suture on the medial side of the implant may be shorter than the segment length of the suture on the lateral side. In implant designs where the bottom surface of the augmentation implant is rough or has protrusions, the implant may be embedded or becomes embedded in the underlying soft tissue (e.g. by subsequent bio-adhesion).

For various relevant implant designs, the following embodiments are optional variations or additional steps that could be performed. The suture may be passed over a pocket at the top side of the implant. The suture may be passed through an underpass tunnel at the bottom side of the implant. The suture may enter or exit the implant through an entrance/exit on the lateral or medial side of the implant.

### BRIEF DESCRIPTION OF THE DRAWINGS

FIGS. 1A and 1B show an example of a prior art surgical procedure for reinforcing a rotator cuff tendon.

FIGS. 2A and 2B show design example #1 for the implant of this invention.

FIGS. 3A and 3B show design example #2 for the implant of this invention.

FIG. 4 demonstrates how length and width are measured for a representative implant.

FIGS. 5A and 5B show design example #3 for the implant of this invention.

FIGS. 6A-6D show design example #4 for the implant of this invention.

FIGS. 7A-7C show design example #5 for the implant of this invention.

FIGS. 8A-8E show an example surgical procedure of this invention.

FIGS. 9A-9D show another example surgical procedure of this invention.

FIGS. 10A and 10B show representative views of a computer simulation using experimental model implants.

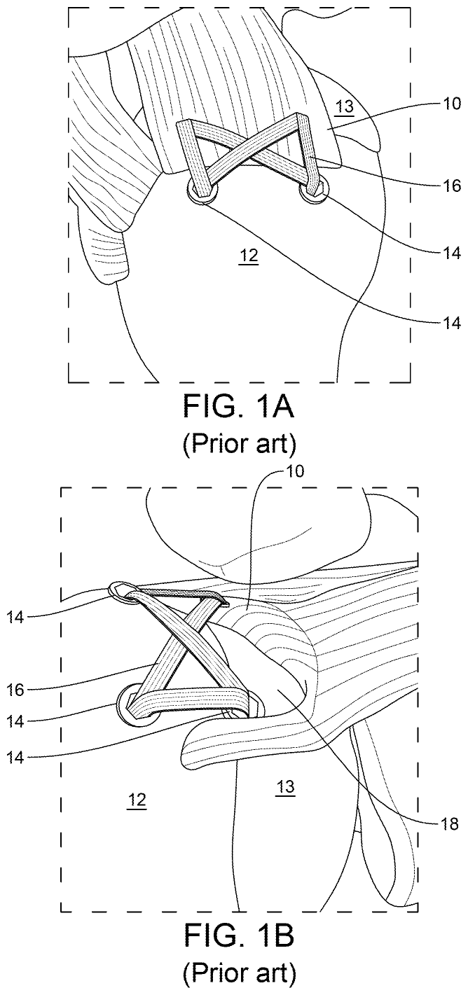

### DETAILED DESCRIPTION

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

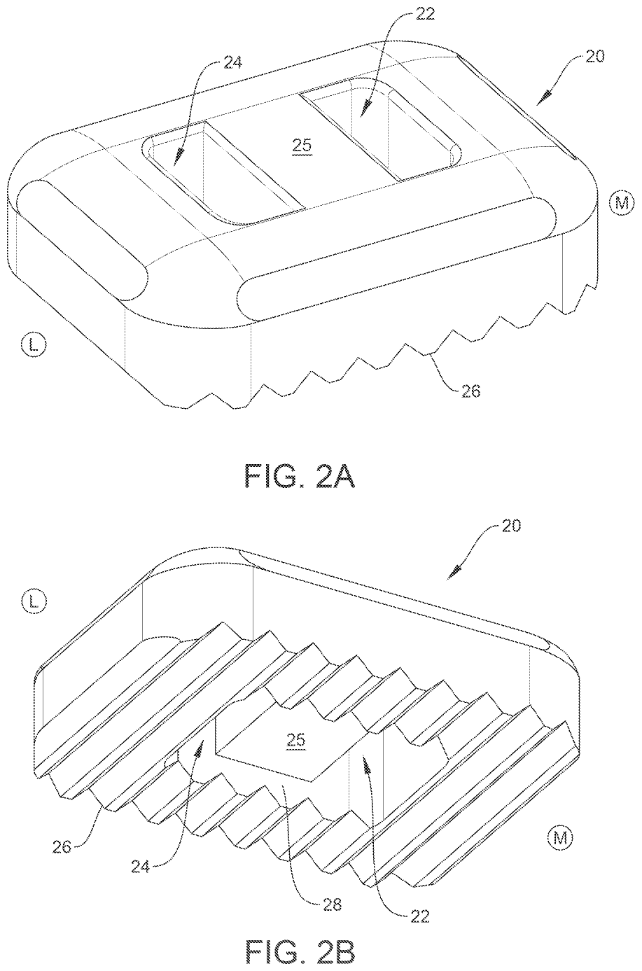

FIGS. 2A and 2B show one possible design (example #1) for the implant of this invention. FIG. 2A shows a top perspective view of implant 20 and FIG. 2B shows a bottom perspective view. As shown in FIG. 2A, implant 20 has a flat rectangular block shape. There are two slots to receive the suture (not shown) for passing through; a medial slot 22 and a lateral slot 24. Slots 22 and 24 are openings through the full thickness of the implant block. In the figures, the medial side is indicated as (M) and the lateral side is indicated as (L). A crossbar 25 separates the two slots. FIG. 2B shows the bottom side of implant 20, which has a series of parallel sawtooth ridges 26 with recessed grooves in between. The purpose of these ridges 26 is to improve traction with the underlying body tissue. Crossbar 25 does not extend the full thickness of the implant block. This creates an underpass tunnel 28 that gives extra room for the suture to pass under.

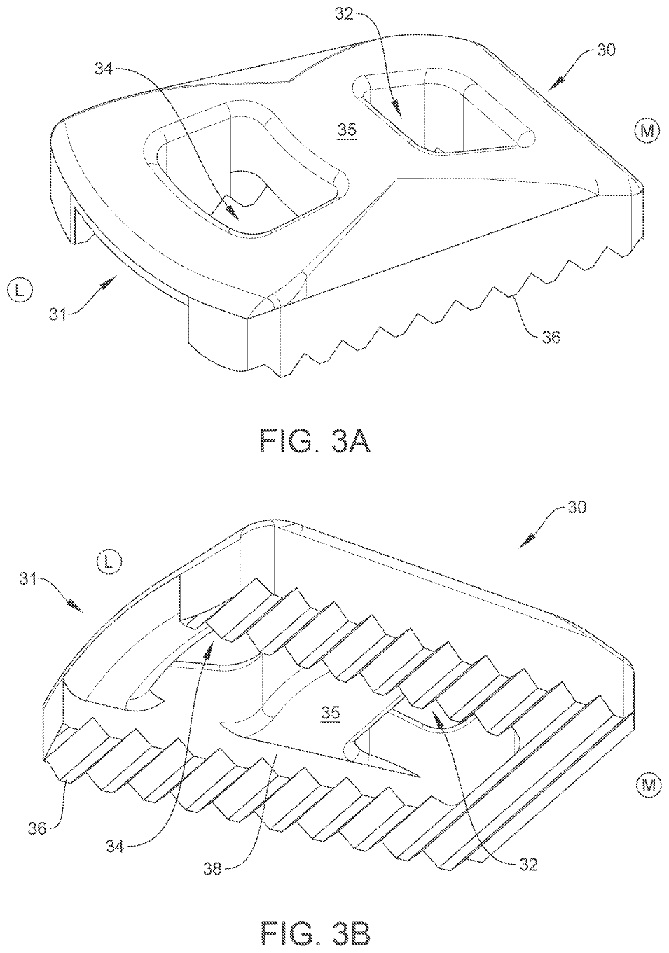

FIGS. 3A and 3B show another possible design (example #2) for the implant of this invention. FIG. 3A shows a top perspective view of implant 30 and FIG. 3B shows a bottom perspective view. There are two slots to receive the suture (not shown) for passing through; a medial slot 32 and a lateral slot 34. Slots 32 and 34 are openings through the full thickness of the implant block. A crossbar 35 separates the two slots. Implant 30 has a slightly trapezoidal shape (from top view). As such, the width of implant 30 becomes narrower going from lateral to medial. FIG. 3B shows the bottom side of implant 30, which has a series of sawtooth ridges 36. This is also different from implant 20 above (design example #1) because underpass tunnel 38 extends from an underpass entrance/exit 31 on the lateral side of implant 30. Underpass tunnel 38 is oriented in a lateral-medial orientation and travels through both slots 32 and 34. Underpass tunnel 38 and entrance/exit 31 gives extra room for passage of the suture taking an underside path into or out of the lateral slot 34.

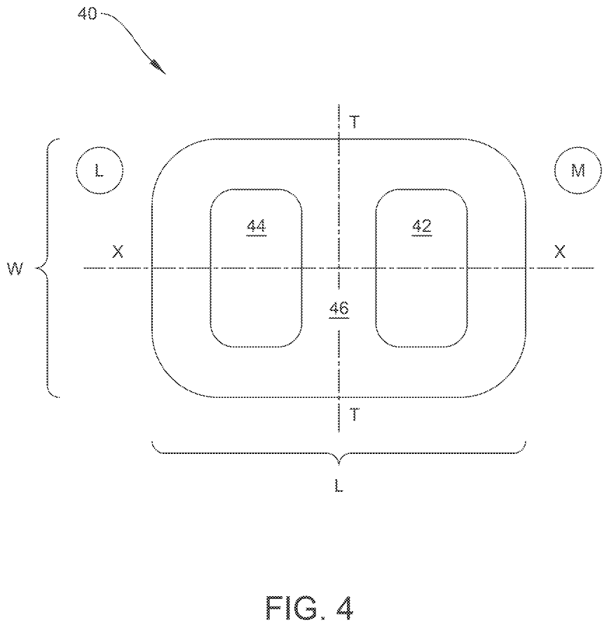

Note also that implant 30 is asymmetric from a lateral-medial comparison. This asymmetric design concept is illustrated in FIG. 4, which shows a representative implant 40 of this invention. It has a medial slot 42 and a lateral slot 44 separated by a crossbar 46. Dashed line T represents an imaginary transverse axis running through the middle of crossbar 46. Transverse axis T splits implant 40 into a medial portion and a lateral portion. If the overall shape (all aspects including top, bottom, and sides) of the lateral portion and the medial portion are mirror images, then implant 40 is medial-lateral symmetric (along the traverse axis). In contrast, if the overall shape of the lateral portion and the medial portion are not mirror images, then implant 40 is medial-lateral asymmetric. FIG. 4 also demonstrates how length and width are measured. Width (W) is measured along the transverse axis T and length (L) is measured along the longitudinal axis represented by the dashed line X.

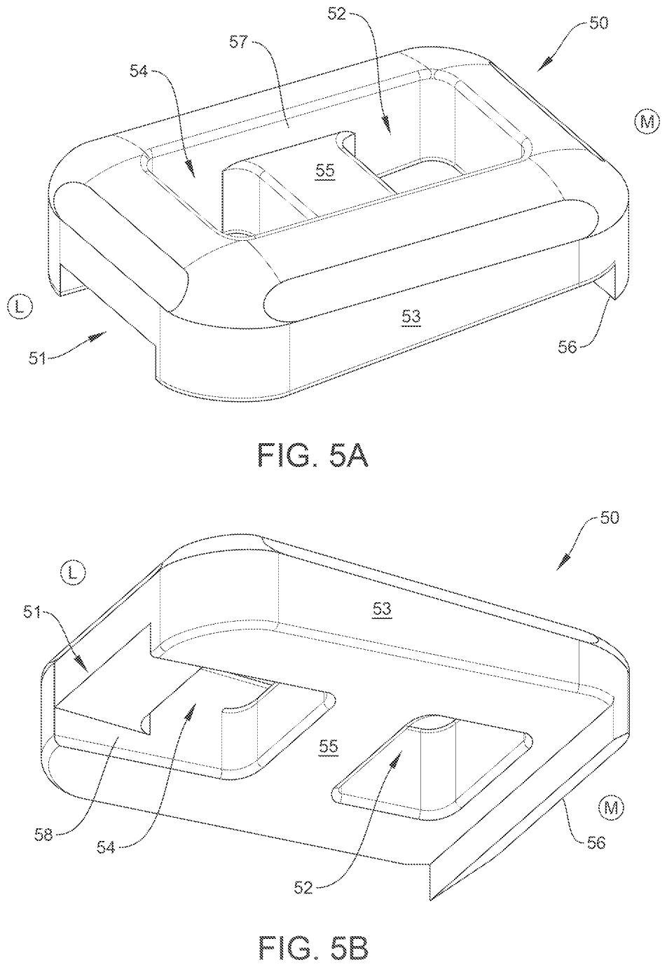

FIGS. 5A and 5B show another possible design (example #3) for the implant of this invention. FIG. 5A shows a top perspective view of implant 50 and FIG. 5B shows a bottom perspective view. There are two slots to receive the suture (not shown) for passing through; a medial slot 52 and a lateral slot 54. A crossbar 55 separate the two slots 52 and 54. Crossbar 55 is depressed relative to the top plane of implant 50. This creates a pocket 57 that makes extra room for the suture to pass overhead crossbar 55, thereby giving the implant assembly a flatter overall profile. An underpass tunnel 58 extends from an underpass entrance/exit 51 on the lateral side of implant 50. On the bottom side of implant 50, there is a single transverse-oriented ledge 56 at the medial edge. The purpose of ledge 56 is to wedge into the underlying body tissue. Note that implant 50 has a tapered shape on the longitudinal side 53 (in side view). The thickness of implant 50 gradually decreases going from lateral to medial. The plane of the bottom side of implant 50 is inclined upward relative to the plane of the top side.

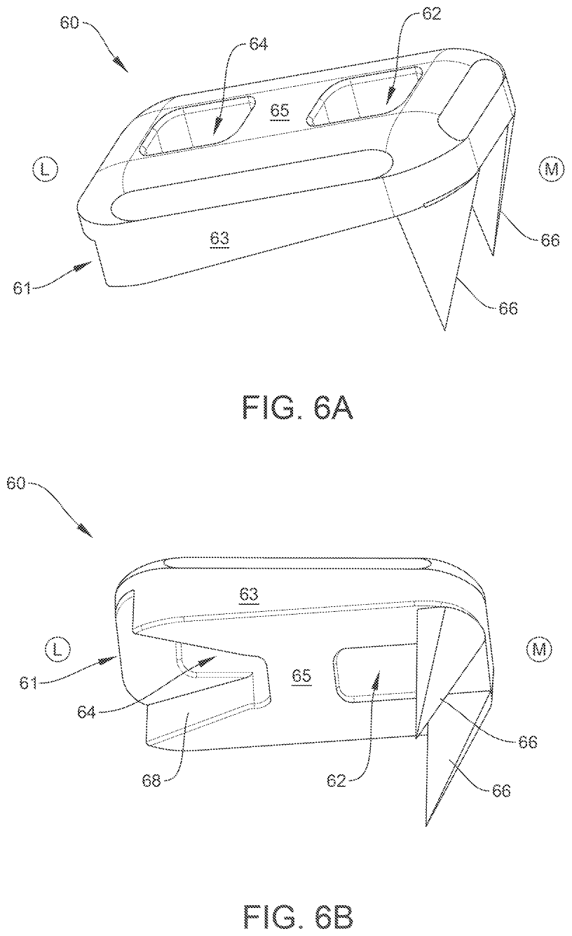

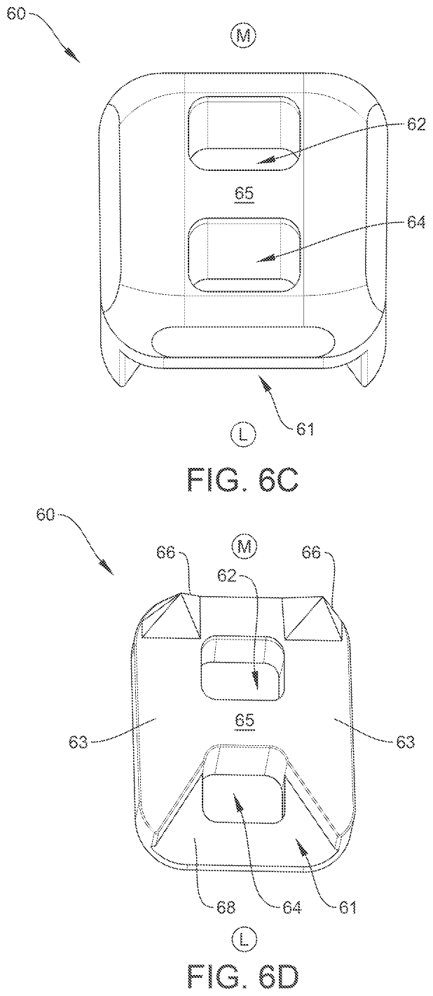

FIGS. 6A-6D show another possible design (example #4) for the implant of this invention. FIG. 6A shows a top perspective view of implant 60 and FIG. 6B shows a bottom perspective view. FIG. 6C shows a top view and FIG. 6D shows a bottom view. There are two slots to receive the suture (not shown) for passing through; a medial slot 62 and a lateral slot 64. A crossbar 65 separate the two slots. On the bottom side, an underpass tunnel 68 extends from an underpass entrance/exit 61 on the lateral side of implant 60 to lateral slot 64. Underpass tunnel 68 flares out (in triangular shape) towards entrance/exit 61. Also, there are two fang-like teeth 66 at the medial edge corners. The purpose of fangs 66 is to bite into the underlying body tissue. Note that implant 60 has a tapered shape on the longitudinal side 63 (in side view). The thickness of implant 60 gradually decreases going from lateral to medial. The plane of the bottom side of implant 60 is inclined upward relative to the plane of the top side. For general overall dimensions, implant 60 is about 6 mm wide, 8 mm long, and 2 mm thick. In an alternate design, fangs 66 could be located at the side edges between slots 62 and 64 at the two locations 63 (see FIG. 6D).

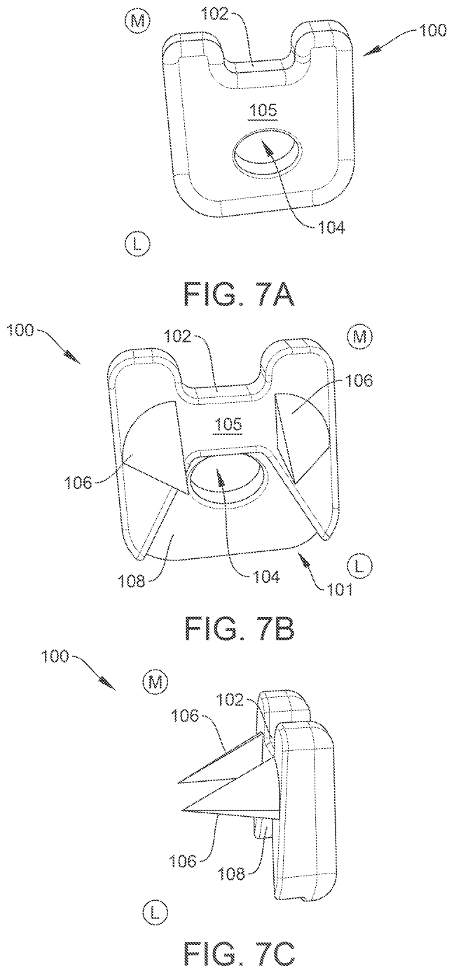

FIGS. 7A-7C show another possible design (example #5) for the implant of this invention. FIG. 7A shows a top perspective view of implant 100; FIG. 7B shows a bottom perspective view; FIG. 7C shows a side perspective view. Implant 100 has only a single round slot 104 to receive the suture (not shown) for passing through. Round slot 104 is located laterally on implant 100. Implant 100 also has a rectangular notch 102 at the medial edge. Notch 102 is located medial to round slot 104. A crossbar 105 separates the slot 104 from notch 102. On the bottom side, an underpass tunnel 108 extends from an entrance/exit 101 on the lateral side. Also, there are two fangs 106 near the side edges between slot 104 and notch 102. In this design, the suture travels up from the anchor site into notch 102, then over crossbar 105, then down through slot 104, then through underpass tunnel 108 and out via entrance/exit 101. During surgical implantation, notch 102 may be positioned so that it is directly over the medial anchor site. This design could be useful for reducing the size of the implant.

FIGS. 8A-8E show an example surgical procedure of this invention for reinforcing a rotator cuff tendon. These figures show the right-side shoulder anatomy from an anterior-posterior view. The left side is lateral and the right side is medial. For the shoulder, seen here is the head of the humerus 70 (bone), supraspinatus tendon 74, and supraspinatus muscle 72 attached thereto. As seen in FIG. 8A, insert two anchors into the humerus bone 70; a lateral-side anchor 78 and a medial-side anchor 76. The site of the medial anchor 76 is under the supraspinatus tendon 74. A suture 75 is pre-fastened to medial anchor 76. Suture 75 has a free end 77. An implant 80 of this invention is ready to be deployed for repairing the rotator cuff tendon. Implant 80 has two slots; medial slot 82 and lateral slot 84. There is an underpass tunnel 86 open on the lateral side of implant 80 that travels to lateral slot 84.

As seen in FIG. 8B, insert free end 77 of suture 75 through medial slot 82 and pass through. Slide implant 80 down along suture 75 towards the implantation site. As seen in FIG. 8C, insert free end 77 of suture 75 through lateral slot 84 and pass through. As seen in FIG. 8D, adjust suture threading through implant 80 so that implant 80 lies flush against tendon 74 at a position where medial slot 82 is directly over medial anchor 76. Adjust suture 75 so that it exits implant 80 out of lateral slot 84 via underpass tunnel 86. As seen in FIG. 8E, fasten free end 77 of suture 75 to the lateral anchor 78. This results in a tight buckle configuration with anchored suture 75 passing up through medial slot 82, down through lateral slot 84, through tunnel 86, and anchored on the lateral side (reference numbers omitted to reduce clutter).

This invention covers all the many possible variations of this technique. For example, the suture may or may not be pre-fastened to the anchor. Or the implant-suture assembly could be fully pre-assembled outside the surgical site and deployed to the surgical site already pre-assembled. Or the implant-suture assembly could be partly pre-assembled (e.g. suture threaded through medial slot only). Or the threading of the suture could take different routes. Or the position of the anchor sites could vary. Or the position or delivery of the implant could vary. Or the order of steps for performing for any of the above could vary.

FIGS. 9A-9D show another example surgical procedure of this invention for reinforcing a rotator cuff tendon. This is the same right-side shoulder setting as above FIGS. 8A-8E, but uses a different implant 90. As repeated from above, seen here is the head of the humerus 70 (bone), supraspinatus tendon 74, and supraspinatus muscle 72 attached thereto. As seen in FIG. 9A, insert two anchors into the humerus bone 70; a lateral-side anchor 78 and a medial-side anchor 76. An implant 90 of this invention is ready to be deployed for repairing the rotator cuff tendon. Implant 90 has two slots; medial slot 92 and lateral slot 94. There is an underpass tunnel 96 under the crossbar between the two slots.

As seen in FIG. 9B, thread free end 77 of suture 75 through medial slot 92 of implant 90. Slide implant 90 down along suture 75 towards the implantation site. Thread free end 77 of suture 75 upwards through lateral slot 94. As seen in FIG. 9C, adjust suture threading through implant 90 so that implant 90 lies flush against tendon 74 at a position between the sites of medial anchor 76 and lateral anchor 78. Tighten suture 75 firmly to fix implant 90 onto tendon 74. As seen in FIG. 9D, fasten free end 77 of suture 75 to lateral anchor 78. This results in a tight buckle configuration with anchored suture 75 passing over the medial edge of implant 90, down through medial slot 92, through underpass tunnel 96, up through lateral slot 94, over the lateral edge of implant 90, and anchored on the lateral side (reference numbers omitted to reduce clutter). As mentioned above, this invention covers all the many possible variations of this technique.

### Experimental Work

Prototype Testing. Various experimental work was performed on prototype implants of this invention. Initial prototype implants were made by 3D printing using polydioxanone (PDO), PLLA (poly-L-lactide), and PCL (polycaprolactone) resin polymers to assess material properties and evaluation on an orthopedic shoulder training model. PDO was found to have insufficient strength and stability over the 6 month desired duration for shoulder repair. Further prototypes were made with PCL/PLLA polymer blend composite with tricalcium phosphate (TCP) because of its longer duration for stability and strength, and also for pH balance. More prototypes were also made by injection molding using high density polyethylene (HDPE) and with resin 3D printing by stereolithography.

Various further benchtop testing was performed on the prototypes. For proof-of-concept that the buckle configuration works to hold and lock the suture, mechanical testing demonstrated that applying a 23 N (newton) load to the suture as buckled into the prototype implants increased suture resistance and hold by 10-fold compared to untensioned sutures. Tensile testing was performed on samples of bovine tendon. The samples were pulled at 90° angle with high loading force and repair failure was determined by measured displacement. With the prototype implants, the tendon samples could withstand at least 300% more loading force before failure as compared to suture alone.

Testing on a human cadaver shoulder demonstrated that implants of this invention could be deployed arthroscopically. In the cadaver shoulder, bone anchors were set onto the medial anchoring site and the suture was buckled into the test implant. The test implant was then delivered through the bore of an arthroscopic cannula having an internal diameter size of 8 mm wide (which is a common bore size). The test implant was set onto the tendon and the lateral site bone anchor was placed. The tendon was pulled into proper position and laid flush on the humeral head bone.

Computer Simulations. Finite element analysis was performed on experimental models of implants of this invention on a 3-dimensional model of the supraspinatus tendon. The test simulated applying a muscle loading onto the supraspinatus tendon for three case scenarios: (1) intact or non-repaired tendon, (2) tendon tear repaired by a conventional double-row, transosseous equivalent technique using tape-type sutures, and (3) tendon tear repaired by a double-row, transosseous equivalent technique that used implant models of this invention. Supraspinatus muscle force was applied on the supraspinatus tendon along the axis parallel to the tendon's laterally running collagen fibers.

Model Construction: Finite element meshes were derived from surface meshes of the full shoulder joint obtained from publicly available databases. Meshes of supraspinatus tendon and humeral head bone were maintained while the remaining portions of the mesh were discarded. The meshes were cleaned, repaired, and scaled to the dimensions of a middle-aged adult male. The material properties for the anatomic model (bone, supraspinatus tendon, and supraspinatus muscle) were acquired from published literature. The supraspinatus tendon and the humeral head bone were tetrahedralized such that both meshes were composed of 104,236 four-noded tetrahedral elements with 9,354 facets. The supraspinatus muscle was represented in a simplified fashion as a long rectangular bar rigidly connected to the supraspinatus tendon.

Simulation Case Scenario #1, Normal Control: A muscle load was applied to orient the shoulder into the plane of the scapula. Following full flexion, shoulder rotation was fixed. Then, 50.1 N (newton) loading force was applied by the supraspinatus muscle for a duration of 1.8 seconds to simulate arm position being held in the plane of the scapula. This simulation established the normal control scenario.

Simulation Case Scenario #2, Conventional Suture Repair: The simulation was repeated for a model of the supraspinatus tendon tear repaired by conventional double-row, transosseous equivalent technique using tape suture. The tape suture was modeled from cylinders reshaped into the material dimensions published for the FiberWire (Arthrex) suture product having 2 mm width and 0.37 mm cross sectional area. The material properties for FiberWire were set based on experimental data. Sutures were fitted through a 2.2 mm cavity in the supraspinatus tendon. The cavity was created to simulate threading of the sutures through the tendon. To replicate the bone screws used as suture anchors, the sutures were rigidly fixed into the humeral head. Again, the simulation was performed with 50.1 N muscle loading force applied to the tendon.

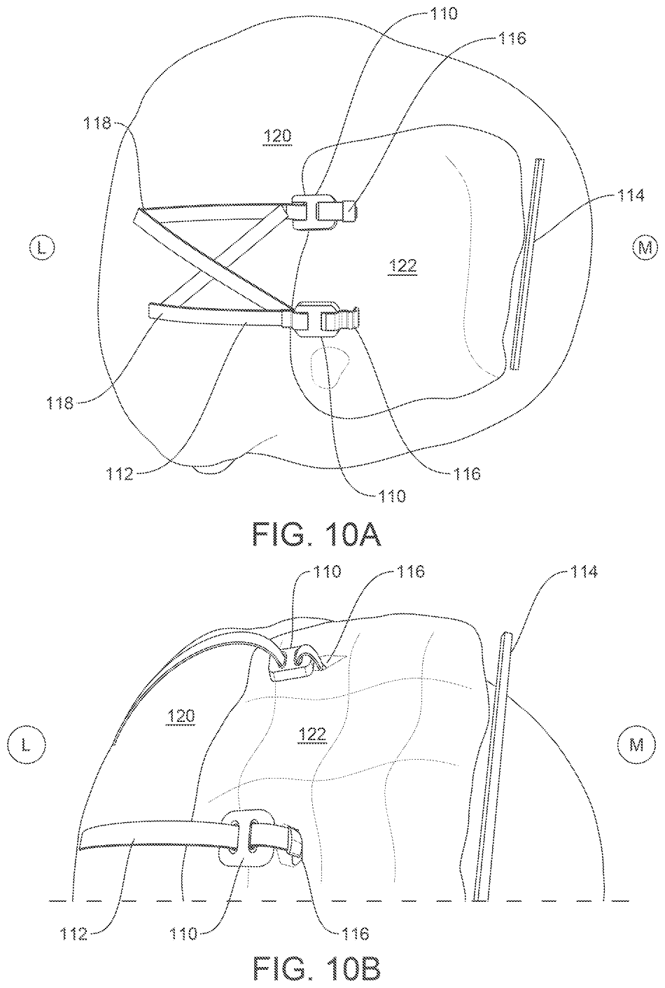

Simulation Case Scenario #3, Implant Repair: This case scenario used the same conditions as above case scenario #2. But instead of the tape suture, experimental models of the implants (tetrahedralized) were inserted. Three different experimental implant models were used: one resembling implant 20 above (example #1, sawtooth ridges), another resembling implant 50 above (example #3, single ledge), and another resembling implant 60 above (example #4, two fangs). Generally, the implant sizes were 4-6 mm wide. One set of model implants were characterized with material properties for a blend of polylactic acid (PLA) and polycaprolactone (PCL). Another set of model implants were characterized with material properties of high-density polyethylene (HDPE). FIGS. 10A and 10B show representative views of this case scenario using the experimental model implants. FIG. 10A is a top view of the shoulder model; FIG. 10B is a perspective view and shows the mesh grid. The anatomical structures are as follows: head 100 of humerus bone, supraspinatus tendon 102, and supraspinatus muscle 104. Two implants 110 are fixed onto supraspinatus tendon 102. Multiple sutures 112 cross the surgical site in double-row configuration. Sutures 112 are anchored at medial anchor site 106 and lateral anchor site 108.

Summary of Simulation Results: As compared to conventional suture threading, rotator cuff repair with the experimental implants reduced the principal stress though the supraspinatus tendon by a factor of 4. Also, in comparison to conventional suture threading, the experimental implants reduced by half the Lagrange strain experienced by the supraspinatus tendon. Further, the experimental implants reduced the loading force exerted at the suture-tendon interface to less than 1 MPa (pascal) and prevented re-tearing or suture pull-through.

Simulation Case Scenario #4, Torn Tendon: This case scenario used the same experimental implant models as above scenario #3 compared against suture only. A crescent-shaped tear at the tendon-bone interface was simulated. In simulated load testing, the experimental implants reduced maximum suture stress by over 50% compared to using suture only. Also, the implants increased the amount of tendon load needed to cause failure by about 300-400% compared to suture alone. Also, there was better distribution of the force load on the tendon and significantly improved contact between bone and tendon. With suture only, many simulation cases resulted in the suture tearing through the tendon. This kind of tearing was not observed in any of the implant cases.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not limited to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof. The terms “first, second, etc.” with respect to elements are being used herein only to distinguish one element from another element. But these are not intended to limit the elements in an ordinal fashion, such as defining the order, position, or priority of the elements.

1. A soft tissue augmentation implant comprising:
a main body;
one or more slots within the main body;
a bottom side and a top side;
wherein the bottom side is more rough than the top side or has one or more protrusions.

2. The augmentation implant of claim 1, further comprising an overpass pocket at the top side.

3. The augmentation implant of claim 1, further comprising an underpass tunnel at the bottom side.

4. The augmentation implant of claim 3, further comprising an entrance/exit on a lateral side, wherein the entrance/exit leads into or out of the underpass tunnel.

5. The augmentation implant of claim 1, wherein the one or more slots consists of two slots.

6. The augmentation implant of claim 5, further comprising a crossbar between the two slots

7. The augmentation implant of claim 1, further comprising a notch on a medial edge side of the main body, and wherein the one or more slots consists of a single slot located laterally to the notch.

8. The augmentation implant of claim 1, further comprising a lateral side and a medial side, and wherein the thickness of the main body is tapered from lateral to medial.

9. The augmentation implant of claim 1, wherein the bottom side has one or more protrusions that are 0.5-5 mm long.

10. The augmentation implant of claim 9, wherein the one or more protrusions are teeth or barbs.

11. A method of surgically reinforcing a target soft tissue in a clinical patient, comprising:
having a soft tissue augmentation implant comprising:
a main body;
one or more slots within the main body;
anchoring a suture at a first anchor site;
passing the suture through the one or more slots;
anchoring the suture at a second anchor site;
securing the augmentation implant on the target soft tissue.

12. The method of claim 11, wherein the second anchor site is located lateral to the first anchor site and at an opposite side of the augmentation implant.

13. The method of claim 11, wherein the suture is pre-fastened to a first suture anchor and wherein the step of anchoring the suture at the first anchor site comprises embedding the first suture anchor at the first anchor site.

14. The method of claim 13, further comprising embedding a second suture anchor at the second anchor site, and wherein the step of anchoring the suture at the second anchor site comprises fastening the suture to the embedded second suture anchor.

15. The method of claim 11, wherein the one or more of the slots of the implant consists of two slots.

16. The method of claim 15, wherein the suture passes through the two slots in a buckle configuration.

17. The method of claim 11, wherein the implant has a bottom side having one or more protrusions, and the method further comprises piercing the one or more protrusions into the target soft tissue.

18. The method of claim 11, wherein the implant further comprises a notch on a medial edge side of the main body, wherein the one or more slots consists of a single slot located laterally to the notch, and the method further comprises inserting the suture into the notch.

19. The method of claim 11, wherein the target soft tissue is a rotator cuff tendon.

20. The method of claim 19, wherein the method is performed arthroscopically at a surgical site for the rotator cuff tendon, and the method further comprises:
inserting a surgical cannula into the surgical site, wherein the surgical cannula has a bore;
deploying the implant through the bore of the surgical cannula.
